(19) United States
(12) Patent Application Publication
(10) Pub. No.: US 2016/0185696 A1
(43) Pub. Date: Jun. 30, 2016

(54) Method for Obtaining Bisphenol-A (BPA) from Polycarbonate Waste Using Microwave Radiation

(71) Applicant: PONTIFICIA UNIVERSIDAD CATOLICA DEL PERU, Lima (PE)

(72) Inventors: Javier Nakamatsu Kuniyoshi, San Isidro, Lima (PE); Bruno Alonso Ortiz Rodriguez, Santiago de Surco, Lima (PE)

(21) Appl. No.: 14/890,722

(22) PCT Filed: Jun. 19, 2013
(86) PCT No.: PCT/PE2013/000004

§ 371 Date: Feb. 17, 2016

(30) Foreign Application Priority Data
May 15, 2013 (PE) 001097-2013/DIN

(51) Int. Cl. C07C 37/56; C07C 37/70

(52) U.S. Cl. CPC C07C 37/56; C07C 37/70

(57) ABSTRACT

The invention consists of a method for obtaining bisphenol-A (BPA) from decomposing polycarbonate waste prepared with bisphenol-A (BPA), which incorporates microwave technology for reaction heating. This method consists of preparing an alkaline solution in alcohol composed of metallic hydroxide in an alcohol, where the hydroxide may be of sodium or potassium, and alcohol is methanol. The solution is mixed with bisphenol-A (BPA) polycarbonate waste and put on a reactor with agitation inside a device provided with a microwave radiation generator for chemical reactions, maintaining a temperature ranging between 90° C. and 150° C., for a period from 3 min to 10 min, preferably between 5 and 7 min, to decompose the polycarbonate and obtain bisphenol-A (BPA).

### 1. FIELD AND PURPOSE OF THE INVENTION

This invention was developed in the field of chemistry, particularly in the area of plastic waste recycling and refers to a method for obtaining bisphenol-A (BPA) from decomposing polycarbonate waste made from that compound through a microwave heating radiation method. This method may be applied in the plastics industry and plastic waste treatment.

2. TECHNICAL PROBLEM AND STATE OF THE ART

In recent years, an environmental problem has been generated by the accumulation of plastic waste among which are the products made from polycarbonate such as CDs, large water containers, cans, baby bottles, safety glass, optical lenses, translucent ceiling panels, among others. This problem has been caused by the intensification of production and consumption of said polymer (in 2005 alone, 3.2 million tons of polycarbonate were produced worldwide), without the implementation of efficient methods of waste recycling. Many of these products contain polycarbonate made from bisphenol-A (BPA).

Furthermore, there are drawbacks referred to the production of bisphenol-A (BPA), also known as 2,2-bis (4-hydroxyphenyl) propane or 4,4′-(propane-2,2-diyl) diphenol, because it is a chemical compound that is currently obtained from petroleum products (non-renewable resource) through a multistep method that generates many by-products.

Among the existing proposals for recycling polycarbonate made from bisphenol-A (BPA) are as follows:

The work of Lian-Chun Hu et al. named Alkali-catalyzed methanolysis of polycarbonate. A study on recycling of bisphenol-A and dimethyl carbonate, describes a polycarbonate, made from bisphenol-A, depolymerization system, which uses a mixture of methanol and toluene, sodium hydroxide and sodium bisulfite as a mean of reaction. To this mixture, virgin polycarbonate pellets are added (3 mm long×1.5 mm diameter cylindrical pieces), which are allowed to react by heating at 40, 50 and 60° C. for periods of 340, 140 and 40 min, respectively. Then, the mixture is poured in cold distilled water or toluene to precipitate the bisphenol-A (BPA) which, once obtained, is filtered. This method reports the recovery of up to 96% of bisphenol-A (BPA).

Furthermore, patent U.S. Pat. No. 7,750,057B2 refers to a method that consists in dissolving the polycarbonate in a chlorinated organic solvent, decomposing it by the action of an alkali metal hydroxide aqueous solution, redissolving it in water, forming two insoluble phases and its subsequent separation. The product is obtained from the aqueous phase.

Likewise, patent 20070185309A1 refers to a method for decomposing polycarbonate using water in a supercritical or subcritical state, at temperatures between 374° C. to 500° C. and pressures from 18 MPa up to 40 MPa.

In a recent publication named: Environmentally friendly chemical recycling of poly (bisphenol-A carbonate) through phase transfer-catalysed hydrolysis under microwave, Tsintzou et al. report the degradation of polycarbonate using microwave radiation. They indicate that, using 5 mL of sodium hydroxide (NaOH) dissolved in water at 5% (w/w) with 0.5 g of polycarbonate in the presence of a catalyst (hexadecyl trimethylammonium bromide), achieves up to 80% degradation of polycarbonate by heating the mixture in a microwave for 40 min at 160° C. Likewise, they report that using sodium hydroxide (NaOH) 10% (w/w), the heating time decreases to 10 min, also at a temperature of 160° C. and in the presence of the catalyst. However, these researchers studied the degradation of polycarbonate, wherein one of the degradation products is bisphenol-A (BPA), but also mentioned other resulting compounds such as phenol, isopropyl phenol and butyl phenol.

### 3. BRIEF DESCRIPTION OF THE INVENTION

The invention is a method for depolymerization (decomposition of polymers into simpler molecules) of polycarbonate waste (FIG. 1) produced from bisphenol-A (BPA) (FIG. 2) in the presence of an alcohol and an alkali at a very short time, including the use of microwave technology.

After depolymerization, the reaction mixture is poured into water to separate the product in high purity, and later isolated by filtration.

### 4. DETAILED DESCRIPTION OF THE INVENTION

The polycarbonate to be depolymerized comes from polycarbonate waste produced from bisphenol-A (BPA), from bottles and CDs, among others, which may contain impurities. First, this material has to be in pieces. Each piece dimension is between 1 mm and 10 mm.

Step (a) consists in preparing an alkaline alcohol solution which contains between 1% and 5% (w/w) alkali, preferably between 2% and 3% (w/w). The alkali can be a metal hydroxide, preferably sodium hydroxide or potassium hydroxide. The alcohol is preferably methanol. Alcohol and alkali should preferably be analytical quality. Furthermore, if preventing coloration in the final product is desired, sodium metabisulfite must be added to the alkaline solution, 0.2% to 0.5% (w/w), preferably 0.3% (w/w) and exchanging the air present in the reactor of step (c) with nitrogen gas.

In step (b), the alkaline solution with the polycarbonate pieces are mixed in the reactor. Each piece is between 1 mm and 10 mm long, although dimensions may increase depending on the reactor size. The mixture ratio is 1 mL to 5 mL of alkaline solution for each gram of polycarbonate, and preferably between 2 mL and 3 mL of solution per gram of polycarbonate.

In step (c), the reactor containing the mixture is sealed and placed in a device with a microwave generator for chemical reactions having temperature and pressure sensors. The device has a microwave radiation generator, preferably with a frequency of 2455 MHz. The reaction is carried out with constant stirring at temperatures between 90° C. and 150° C., preferably 110° C. to 120° C.; and at pressures between 0.1 MPa and 1.2 MPa, for a period between 3 and 10 min, preferably between 5 and 7 min. During this process, polycarbonate is decomposed to bisphenol-A (BPA). At the end of this period, the reaction mixture is cooled, reaching temperatures between 45° C. and room temperature.

In step (d), the impurities or other insoluble materials present in the reaction mixture of (c) are separated by a filtration process. This filtration can be performed with grade 1 (11 μm) filter paper. The filtrate is poured into water, using a volume of water 5 to 20 times the volume of the reaction from the alkaline solution with stirring for 10 to 30 min at room temperature. This causes precipitation of bisphenol-A (BPA). Finally, in step (e) the mixture is allowed to stand for 30 min, filtered with grade 1 filter paper to recover bisphenol-A (BPA) in the solid state and allowed to dry. Filtration processes may be performed under vacuum.

Bisphenol-A (BPA) obtained by the polycarbonate decomposition above-described has a melting point of 156° C. (similar to the scientific literature); presents infrared spectrum (FIG. 3) and NMR both hydrogen-1, ¹H-NMR (FIG. 4) and carbon-13, ¹³C-NMR (FIG. 5), in dimethyl sulfoxide (DMSO) as solvent equal to those reported in the scientific literature. Moreover, the mass spectrum (electrospray ionization and ion trap detector) of the product obtained corresponds to that expected for the BPA (FIG. 6). The peak (M-H)−, m/z of 227.2, and a peak at m/z of 455.1 corresponding to (2M-H)− can be observed, equivalent to the association of two molecules of BPA at a loss of a proton.

The polycarbonate to be depolymerized can be virgin polycarbonate made from bisphenol-A (BPA).

Methods above-described as state of the art, have disadvantages with respect to this invention in the following aspects:

The method reported by Hu et al requires the use of toluene as co-solvent in the reaction which is toxic and carcinogenic. In this invention, only methanol (an economical and ecofriendly solvent) is used; and also, according to Hu, the heating time required for the reaction ranges from 40 min to 340 min, far more than 3 min to 10 min of the proposed method. Furthermore, the recovery yields of bisphenol-A (BPA) by the proposed method reach 90%, which is comparable to the method of Hu (96%), if the use of samples of virgin polycarbonate, instead of waste from polycarbonate (CDs and large water containers) is taken into account, therefore, this performance is not reached without the presence of virgin polycarbonate. Moreover, the method described by Hu does not include the use of microwave generator.

The method of U.S. Pat. No. 7,750,057B2 uses a considerable amount of a chlorinated organic solvent (toxic and eco-unfriendly) to dissolve the polycarbonate and a highly concentrated metal hydroxide solution (45% to 55%, w/w). Our proposed method uses low concentration (2.2%) of sodium hydroxide (NaOH). Furthermore, said method uses several hours (over 10 hours), while our proposed method uses less than 10 min. Moreover, the method described in this patent does not include the use of a microwave generator.

While the patent US20070185309A1 does not require the use of organic solvents as the previous methods using only water as a solvent, it must be in (or close to) its critical point (374° C. and 22.06 MPa). These conditions require the use of special and expensive equipment. Moreover, as reported in the same patent, given the extreme conditions, although the reaction time is very short (1 to 5 min), it is crucial to timely stop the reaction because by-products are generated quickly. In comparison, the proposed method requires much lower pressures, of around 0.7 MPa. Furthermore, the method described in this patent does not include the use of a microwave generator.

The method of Tsintzou et al. uses aqueous solutions of sodium hydroxide as reaction medium, but requires the use of a phase transfer catalyst. That study showed that without the presence of the catalyst (hexadecyl trimethylammonium bromide), polycarbonate degradation is very limited (less than 25%). Regarding the concentration of sodium hydroxide, the results show that a minimum of 5% concentration (w/w) is required to complete the reaction in 40 min at 160° C.; if the alkali concentration increases to 10% (w/w), the reaction time is reduced to 10 min at 160° C. In comparison, the proposed method uses methanol instead of water, a lower alkali concentration of 5% (w/w), a lower temperature (120° C.) and a shorter reaction time (5 min). Moreover, the use of a phase transfer catalyst is not required. Another relevant difference is the ratio between the polycarbonate and the reaction medium. In the method of Tsintzou et al, 10 parts of the reaction medium (alkaline solution) with a part of polycarbonate (10:1) were used. In the proposed method, the ratio is 2:1. An important point to consider is that the results of procedures of Tsintzou et al, consist in 80% polycarbonate degradation (leading to the formation of oligomers and around 60% bisphenol-A) and not 90% bisphenol-A (BPA) yields, as reported in the proposed method.

Therefore, the advantages of the proposed method are summarized as follows:
Avoids the use of organic solvents derived from non-renewable and harmful resources for the environment, because only methanol in the alkaline solution and water in the filtration step are used.
Substantially reduces the reaction time (between 3 and 10 min).
Achieves high yields without the formation of important by-products.
The method is not affected if impurities are present in the polycarbonate, for example, in CDs.

### 5. EMBODIMENT

### Embodiment 1

Polycarbonate from CDs.

In a glass reactor, 0.0250 g of sodium metabisulfite were added. 10 mL of a sodium hydroxide in methanol solution (2.2% w/w sodium hydroxide) were added. The methanol was of analytical quality. A magnetic stir bar coated with polytetrafluoroethylene (PTFE) was used for stirring. Finally, 5.0158 g of polycarbonate from CDs were added (pieces with dimensions between 1 and 10 mm). The air inside the reactor was displaced using a stream of nitrogen before closing. The (sealed) reactor was placed in a microwave generator, heated at 120° C., maintaining that temperature for 10 min and cooled down to 45° C. before removing the reactor from the generator. Upon removal of the reactor from the generator, the complete disappearance of the pieces of polycarbonate and the presence of a burgundy colored solution were observed. This solution was vacuum filtered with a grade 1 (11 μm) filter paper, retained remaining solid (mainly excess sodium hydroxide in solid state and other materials present in CDs). The filtrate was poured into 200 mL of distilled water at room temperature with stirring for 30 min. Precipitating bisphenol-A (BPA) was observed as a white solid. Then, the solution was allowed to stand for 30 min before it is again vacuum filtered, with a grade 1 filter paper to recover the bisphenol-A (BPA). The filtrate was allowed to dry. The yield obtained from the reaction was 89.7% (w/w). The product melting point was 156° C.

### Embodiment 2

Polycarbonate from CDs.

The reaction was performed identically to embodiment 1 with a reaction time of 3 min at 120° C. The yield achieved was 88.5% (w/w).

### Embodiment 3

Polycarbonate from large water containers.

The reaction was performed similarly to embodiment 1 but using large water containers (made from polycarbonate) cut into pieces with equal dimensions. The yield achieved was 92.6% (w w).

### Embodiment 4

Polycarbonate from large water containers at lower temperature.

The reaction was conducted similarly to embodiment 2 but limiting the temperature to 90° C. The yield achieved was 68.6% (w/w).

### Embodiment 5

Polycarbonate from CDs with ethanol.

The reaction was performed similarly to embodiment 1 but replacing methanol with ethanol. Upon completion of the reaction, unreacted remains of polycarbonate were observed, and upon pouring the mixture over distilled water, a non-aqueous phase liquid (diethyl carbonate) was formed and no precipitation of bisphenol A (BPA) was observed in this system.

### Embodiment 6

Polycarbonate from CDs with ethanol and more reaction time.

The reaction was performed similarly to embodiment 1 by replacing methanol with ethanol and a reaction time of 10 min at 150° C. Upon completion of the reaction, no polycarbonate remnants were observed, but upon pouring the mixture filtered over distilled water, a second phase was formed and precipitation of bisphenol-A (BPA) in this system was not observed.

Therefore, according to embodiment 5 and embodiment 6, methanol compared to ethanol, was the most appropriate solvent for obtaining bisphenol-A (BPA) in the process of the invention.

### Embodiment 7

Polycarbonate from CDs with ethylene glycol.

In a glass reactor, 0.0250 g of sodium metabisulfite were added. 10 mL of a sodium hydroxide solution in ethylene glycol (2.2% w/w sodium hydroxide) were added. The ethylene glycol was of analytical quality. A magnetic stir bar coated with polytetrafluoroethylene (PTFE) was added for stirring. Finally, 5.0188 g of polycarbonate from CDs were added (pieces with dimensions between 1 and 10 mm). The air inside the reactor was displaced using a stream of nitrogen before closing. The (sealed) reactor was placed in a microwave generator, heated at 120° C., maintaining that temperature for 10 min and cooled down to 45° C. before removing the reactor from the generator. Upon removal of the reactor from the generator, the complete disappearance of the pieces of polycarbonate was not observed. The reactor was placed back into the microwave generator, heated at 150° C., maintaining that temperature for 10 min and then cooled down to 45° C. Upon removal of the reactor from the generator, polycarbonate solid remnants were still observed. This solution was vacuum filtered with a grade 1 (11 μm) filter paper, retained the remaining solid (mainly excess polycarbonate). The filtrate was poured into 200 mL of distilled water at room temperature with stirring for 30 min. Precipitating bisphenol-A (BPA) was observed, as a white solid. Then, the solution was allowed to stand for 30 min before it is again vacuum filtered, with a grade 1 filter paper to recover the bisphenol-A (BPA). The filtrate was allowed to dry. The yield obtained from the reaction was 21.4% (w/w).

### Embodiment 8

Polycarbonate from CDs with water.

The reaction performance was similar to embodiment 7 but replacing ethylene glycol with water and the second heating cycle at 180° C. instead of 150° C. After the first heating cycle, no changes in the polycarbonate pieces were observed. In the second cycle, the limit set pressure of 3 MPa was reached, therefore, the reaction finished after around 5 min. The yield achieved was only 41%.

Therefore, according to embodiment 7 and embodiment 8, the use of both ethylene glycol and water compared to methanol, does not exceed 41% yield to obtain bisphenol-A (BPA).

### 6. BRIEF DESCRIPTION OF FIGURES

FIG. 1: Chemical structure of polycarbonate made from bisphenol-A (BPA).

FIG. 2: Chemical structure of bisphenol-A (BPA).

FIG. 3: Infrared spectrum of the product obtained (KBr disc).

FIG. 4: NMR spectrum, ¹H-NMR (300 MHz), of the product obtained (in dimethylsulfoxide-d₆ DMSO as solvent).

FIG. 5: NMR spectrum, ¹³C-NMR (75 MHz), of the product obtained (in dimethylsulfoxide-d₆ DMSO as solvent).

FIG. 6: Mass spectrum (electrospray ionization and ion trap detector) of the product obtained.

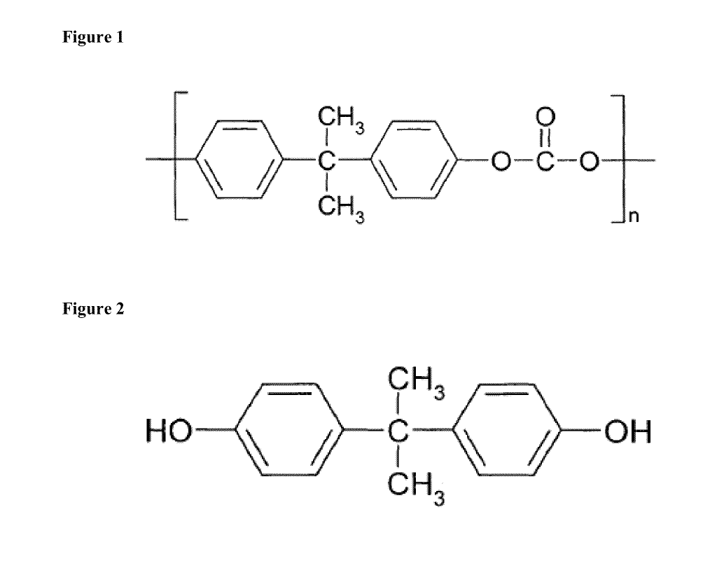

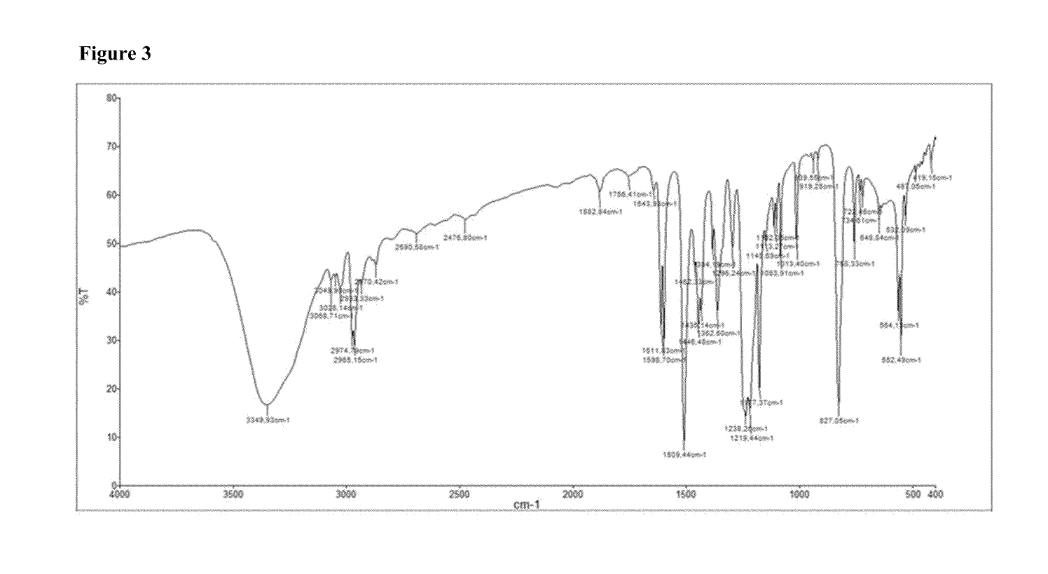

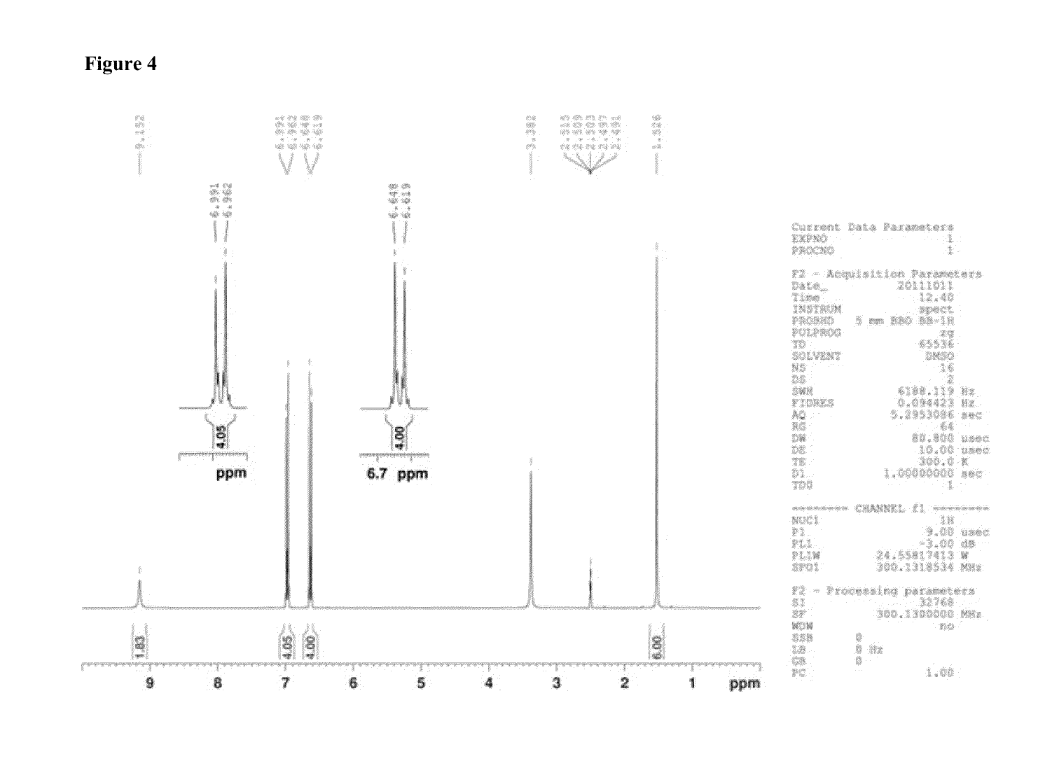

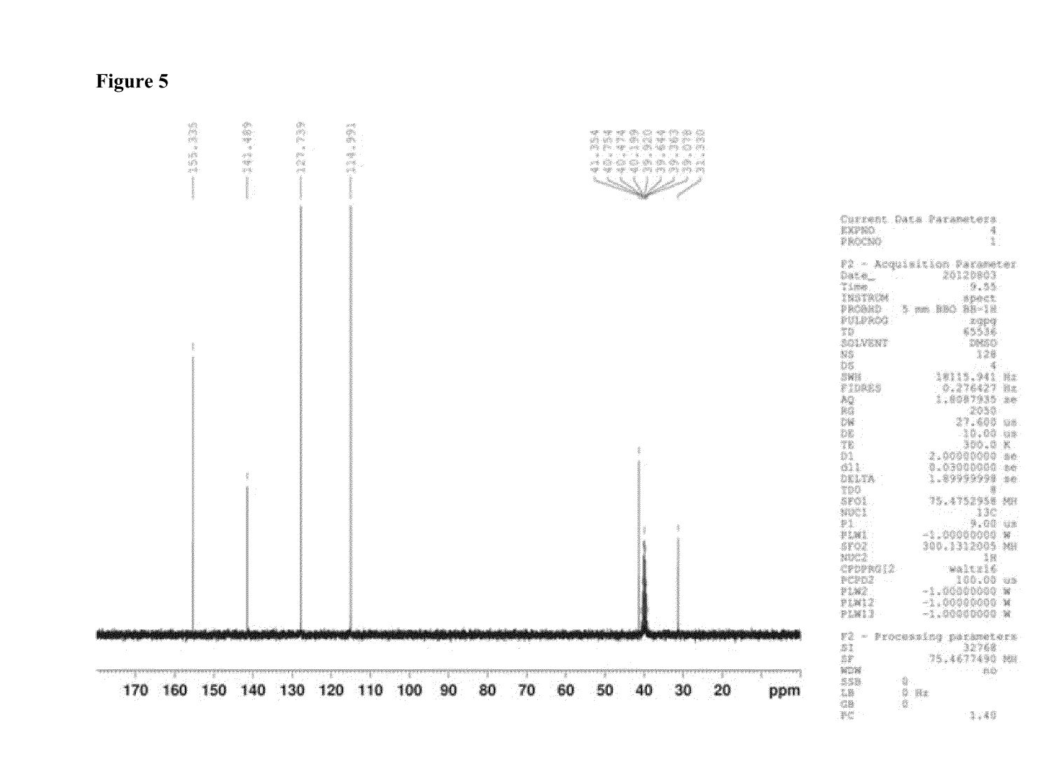

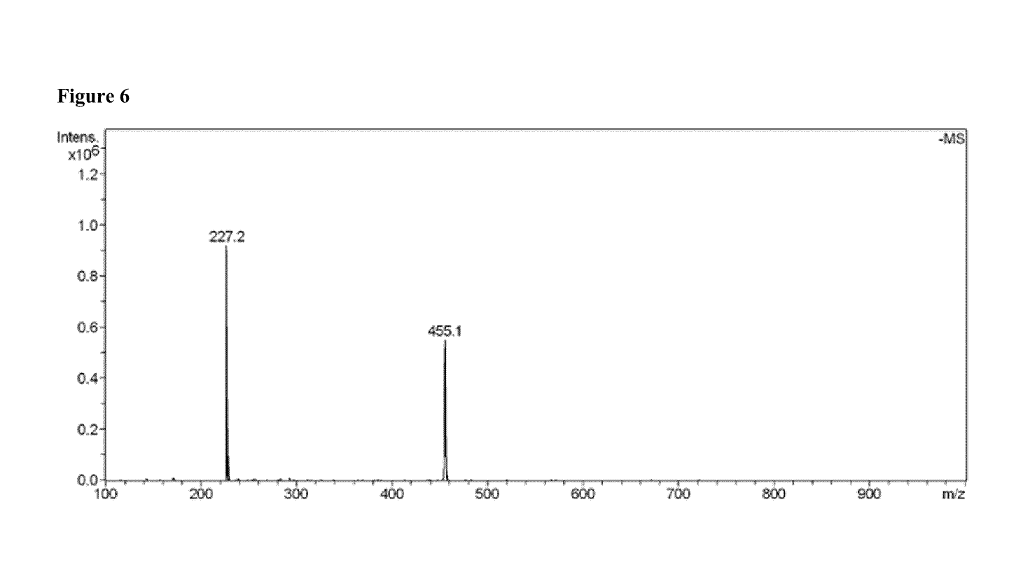

1. Method for decomposition of polycarbonate waste from bisphenol-A (BPA), characterized as follows:
a) Prepare an alkaline solution in alcohol, containing between 1% and 5% (w/w) of alkali, and methanol;
b) Combine the alkaline solution with polycarbonate waste in a reactor, where the ratio of the mixture is from 1 mL to 5 mL of alkaline solution per gram of polycarbonate waste;
c) Tightly close the reactor with the alkaline solution and put it inside a device provided with a microwave radiation generator with a frequency of 2455 MHz, where the reaction is made with constant agitation at a temperature between 90° C. and 150° C. and a pressure between 0.1 MPa and 1.2 MPa for a period from 3 min to 10 min, for polycarbonate decomposition; and, at the end of this period, the reaction mixture is allowed to cool down until reaching a temperature between 45° C. and room temperature;
d) Filter this mixture with grade 1 (11 μm) filter paper, and then pour it into water using a water volume from 5 to 20 times the alkaline solution from the reaction, agitating from 10 min to 30 min at room temperature; and
e) Allow the mixture to stand for 30 min; filter it again to recover bisphenol-A (BPA) in solid state and allow to dry.

2. Method for decomposition of polycarbonate waste from bisphenol-A (BPA) according to claim 1, characterized because in step a) alkali is preferably between 2% and 3% (w/w).

3. Method for decomposition of polycarbonate waste from bisphenol-A (BPA) according to the claims above, characterized because in step a) alkali is a metallic hydroxide, preferably sodium hydroxide or potassium hydroxide.

4. Method for decomposition of polycarbonate waste from bisphenol-A (BPA) according to the claims above, characterized because in step b) the ratio of mixture of alkaline solution with polycarbonate waste preferably is between 2 mL and 3 mL of solution per gram of polycarbonate waste.

5. Method for decomposition of polycarbonate waste from bisphenol-A (BPA) according to the claims above, characterized because in step c) the temperature is preferably between 110° C. and 120° C.

6. Method for decomposition of polycarbonate waste from bisphenol-A (BPA) according to the claims above, characterized because in step c) time is preferably between 5 and 7 min.

7. Method for decomposition of polycarbonate waste from bisphenol-A (BPA) according to the claims above, characterized because in step d) filtering processes are conducted in vacuum.

8. Method for decomposition of polycarbonate waste from bisphenol-A (BPA) according to the claims above, characterized because in step a) between 0.2% and 0.5% (w/w) of sodium metabisulfite is incorporated into the alkaline solution in alcohol; and, in step c) air is replaced in the nitrogen reactor.

9. Method for decomposition of polycarbonate waste from bisphenol-A (BPA) according to the claims above, characterized because in step a) preferably 0.3% (w/w) of sodium metabisulfite is incorporated into the alkaline solution in alcohol.

10. Method for decomposition of polycarbonate waste according to any of the claims above, characterized because waste may be replaced by pure or virgin bisphenol-A (BPA) polycarbonate.
